(19) United States
(12) Patent Application Publication
(10) Pub. No.: US 2021/0100744 A1
(43) Pub. Date: Apr. 8, 2021

(54) BIORESPONSIVE HYDROGEL MATRIXES AND METHODS OF USE

(71) Applicant: NORTH CAROLINA STATE UNIVERSITY, Raleigh, NC (US)

(72) Inventor: Zhen Gu, Los Angeles, CA (US)

(21) Appl. No.: 17/054,609

(22) PCT Filed: May 13, 2019
(86) PCT No.: PCT/US2019/031971

§ 371 Date: Nov. 11, 2020

Related U.S. Application Data
(60) Provisional application No. 62/670,632, filed on May 11, 2018.

(51) Int. Cl. A61K 9/06; A61K 9/00; A61K 47/34; A61K 47/42; C07K 16/28; A61K 39/395; A61K 45/06; A61P 35/00

(52) U.S. Cl. CPC A61K 9/06; A61K 9/0019; A61K 47/34; A61K 47/42; A61K 2039/505; A61K 39/39541; A61K 45/06; A61P 35/00; C07K 2317/76; C07K 16/2827

(57) ABSTRACT

Disclosed are compositions and methods for treating cancer with a hydrogel matrix comprising a chemotherapeutic agent and a blockade inhibitor. Disclosed herein are bioresponsive hydrogel matrixes comprising a reactive oxygen species scavenger.

This application claims the benefit of U.S. Provisional Application No. 62/670,632, filed on May 11, 2018, which is incorporated herein by reference in its entirety.

### I. BACKGROUND

Immune checkpoint blockade (ICB) targeting the programmed death-1/programmed death-ligand 1 (PD-1/PD-L1) pathway induces remarkable clinical responses in various malignancies, including melanoma, non-small cell lung, kidney, head and neck and bladder cancers. However, only patients with immunogenic tumors characterized by high neoantigen burden, pre-infiltration of effector T cells and expression of PD-L1 seem to achieve durable clinical responses after the administration of ICB. Moreover, clinical application of ICB has also been associated with various side effects in normal organs. Based on these studies, strategies aimed at promoting an immunogenic tumor phenotype, increasing ICB response, and avoiding severe side effects remain a central theme in the field of cancer immunotherapy. What are needed are new cancer therapies and treatment strategies that can

### II. SUMMARY

Disclosed are methods and compositions related to bioresponsive hydrogel matrixes.

In one aspect, disclosed herein are bioresponsive hydrogel matrixes comprising a reactive oxygen species scavenger (such as, for example, L-Methionine, sodium pyruvate; mannitol; sodium azide; uric acid; Ebselen; 6-hydroxy-2,5,7,8-tetramethylchroman-2-carboxylic acid (Trolox); 4,5-dihydroxybenzene-1,3-disulfonate (Tiron); α-tocopherol (Vitamin E); 2-(4-Carboxyphenyl)-4,4,5,5-tetramethylimidazoline-1-oxyl-3-oxide (Carboxy-PTIO); manganese(III)-tetrakis(4-benzoic acid)porphyrin (MnTBAP), acetyl-L-cysteine; Vitamin A; Vitamin C; Glutathione; and/or, Beta-carotene) and an inhibitor of indoleamine-2,3-dioxygenase (IDO) (such as, for example, dextro-1-methyl tryptophan (D-1MT also known as indoximod), NLG919, BMS-986205, norharmane, rosmarinic acid, epacadostat, INCB024360 analogue, IDO inhibitor 1, PF-06840003, and/or navoximod).

In one aspect, the bioresponsive hydrogel matrix of any preceding aspect can be formulated as a triblock copolymer or multiblock copolymer (for example a triblock copolymer wherein the triblock copolymer comprises polyethylene glycol flanked by a polypeptide block comprising the reactive oxygen species scavenger and an inhibitor of indoleamine-2,3-dioxygenase (IDO).

Also disclosed herein are bioresponse hydrogel matrixes of any preceding aspect further comprising an immune blockade inhibitor (including, but not limited to a PD-1/PD-L1 blockade inhibitor such as, for example, nivolumab, pembrolizumab, spartalizumab, pidilizumab, atezolizumab, avelumab, durvalumab, and BMS-9365599 and/or a CTLA-4 inhibitor such as, for example, ipilimumab).

In one aspect, the bioresponsive hydrogel matrix of any of preceding aspect can further comprises a chemotherapeutic agent.

Also disclosed herein are method of treating a cancer in a subject comprising administering to the subject the bioresponsive hydrogel matrix of any preceding aspect. For example, disclosed herein are methods of treating a cancer in a subject comprising administering to the subject a bioresponsive hydrogel matrix comprising a reactive oxygen species scavenger (such as, for example, L-Methionine, sodium pyruvate; mannitol; sodium azide; uric acid; Ebselen; 6-hydroxy-2,5,7,8-tetramethylchroman-2-carboxylic acid (Trolox); 4,5-dihydroxybenzene-1,3-disulfonate (Tiron); a-tocopherol (Vitamin E); 2-(4-Carboxyphenyl)-4,4,5,5-tetramethylimidazoline-1-oxyl-3-oxide (Carboxy-PTIO); manganese(III)-tetrakis(4-benzoic acid)porphyrin (MnTBAP), acetyl-L-cysteine; Vitamin A; Vitamin C; Glutathione; and/or, Beta-carotene) and an inhibitor of indoleamine-2,3-dioxygenase (IDO) (such as, for example, dextro-1-methyl tryptophan (D-1MT also known as indoximod), NLG919, BMS-986205, norharmane, rosmarinic acid, epacadostat, INCB024360 analogue, IDO inhibitor 1, PF-06840003, and/or navoximod).

In one aspect, disclosed herein are methods of treating a cancer of any preceding aspect, wherein the bioresponsive hyrodrogel matrix further comprises an immune blockade inhibitor (including, but not limited to a PD-1/PD-L1 blockade inhibitor such as, for example, nivolumab, pembrolizumab, spartalizumab, pidilizumab, atezolizumab, avelumab, durvalumab, and BMS-9365599 and/or a CTLA-4 inhibitor such as, for example, ipilimumab).

Also disclosed herein are methods of treating a cancer of any preceding aspect, wherein the bioresponsive hyrodrogel matrix further comprises a chemotherapeutic agent.

In one aspect, disclosed herein are methods of treating a cancer of any preceding aspect, wherein the hydrogel matrix comprises releases the inhibitor of IDO, the immune blockade inhibitor, the chemotherapeutic agent, or any combination thereof into the tumor microenvironment upon exposure to reactive oxygen species (ROS).

Also disclosed herein are methods of treating a cancer of any preceding aspect, wherein the hydrogel releases the chemotherapeutic and PD-1/PD-L1 blockade inhibitor into the tumor microenvironment for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In one aspect, disclosed herein are methods of treating a cancer of any preceding aspect, wherein the cancer is a cancer with low PD-L1 expression or a non-immunogenic cancer selected from the group consisting of melanoma, non-small cell lung carcinoma, urothelial cancer, renal cancer, head and neck cancer, Hodgkin's lymphoma, and bladder cancer.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A and 1B show schematics of bio-stimuli triggered aPD-L1 and D-1MT localized delivery based on the injectable thermo-sensitive hydrogel for tumor microenvironmental regulation and immunotherapy. FIG. 1A shows the structure of P(Me-D-1MT)-PEG-P(Me-D-1MT) and ROS triggered polymeric hydrophobicity transition. FIG. 1B shows a schematic illustration of localized hydrogel formation and bio-stimuli triggered drug release and synergistic immunotherapy.

FIG. 2 shows synthesis routes of dextro-1-methyltryptophan (D-1MT) NCA (a), L-Methionine NCA (b) and P(Me-D-1MT)-PEG-P(Me-D-1MT) (c).

FIGS. 3A, 3B, 3C, 3D, 3E, 3F, 3G, 3H, 3I, and 3J. Function characterization of the P(Me-D-1MT)-PEG-P(Me-D-1MT)-based hydrogel. FIG. 3A shows the size distribution and TEM image of P(Me-D-1MT)-PEG-P(Me-D-1MT)-formed micelles in water (scale bar: 200 nm). FIG. 3B shows the sol-to-gel transition phase diagram of P(Me-D-1MT)-PEG-P(Me-D-1MT) with the concentration from 4.0 wt % to 10.0 wt % and SEM image of lyophilized gel at the concentration of 8.0 wt % (scale bar: 10 μm), respectively. FIG. 3C shows the rheology test of the P(Me-D-1MT)-PEG-P(Me-D-1MT)-formed hydrogel with the concentration of 8.0 wt % (a), 12.0 wt % (b) and IgG-loaded hydrogel (8.0 wt %) (c). FIG. 3D shows photographs of the sol-to-gel transition with the increasing of temperature (a and b), the gel disintegration by incubating with H₂O₂ (c) and injectable gelation test in water at 37° C. (d). FIG. 3E shows CD spectra of P(Me-D-1MT)-PEG-P(Me-D-1MT) (a) and P(Me-D-1MT)-PEG-P(Me-D-1MT) oxide (b) with the concentration of 0.1 mg/mL. FIG. 3F shows In vitro degradation behavior of the 8.0 wt % P(Me-D-1MT)-PEG-P(Me-D-1MT) hydrogel when incubated with (a) tris-HCl buffer solution (pH=7.4), (b) 2.0 mM H₂O₂ in tris-HCl buffer solution (pH=7.4) and (c) 5.0 U/mL Proteinase K in tris-HCl buffer solution (pH=7.4) (n=3). FIG. 3G shows In vivo degradation behavior and tissue biocompatibility of the in-situ-formed hydrogel (8.0 wt %) with H&E staining of the surrounding skin at different testing time (Scale bar: 400 μm). FIG. 3H shows H₂O₂-triggered (10.0 mM) hydrogel (8.0 wt %) degradation behavior in PBS (pH=7.4) (n=3). FIG. 3I shows H₂O₂-triggered (10.0 mM) IgG release behavior in PBS (pH=7.4) (n=3). FIG. 3J shows the in vitro H₂O₂ scavenging test incubated without (a) or with (b) P(Me-D-1MT)-PEG-P(Me-D-1MT)-based hydrogel (8.0 wt %) in PBS (pH=7.4) (n=3). Data depict mean±s.d.

FIGS. 4A, 4B, 4C, 4D, 4E, and 4F show the antitumor efficiency evaluation in vivo. FIG. 4A shows the intratumoral drug retention behavior after treated with both of free aPD-L1 and aPD-L1-loaded P(Me-D-1MT)-PEG-P(Me-D-1MT)-based hydrogel after different interval in vivo (Scale bar: 50 μm). FIG. 4B shows the in vivo bioluminescence imaging of the B16F10 tumors which observed at the designed testing points, (4C) quantified individual tumor growth carves, (4D) average tumor volumes (n=5), (4E) average body weights (n=5) and (4F) survival carves (n=5) with the single treatment of various therapeutics (G1, PBS; G2, blank P(Me-D-1MT)-PEG-P(Me-D-1MT) hydrogel; G3, Free D-1MT and aPD-L1; G4, aPD-L1 loaded P(Me-D-1MT)-PEG-P(Me-D-1MT)) when the tumor volumes reached to ˜110 mm³ on the 7^{th} day (marked by red arrow). Data depict mean±s.d., values were analyzed by one-way ANOVA with Tukey's post hoc test for 4D and by log-rank (Mantel-Cox) test for 4F, *P<0.05, **P<0.01, ***P<0.001.

FIGS. 5A, 5B, 5C, 5D, 5E, 5F, 5G, and 5H show the In vivo antitumor immune response investigation after treated with various treatments (G1, PBS; G2, blank P(Me-D-1MT)-PEG-P(Me-D-1MT) hydrogel; G3, Free D-1MT and aPD-L1; G4, aPD-L1 loaded P(Me-D-1MT)-PEG-P(Me-D-1MT)). FIG. 5A shows representative immunofluorescence of tumors exhibited CD8+ T cells infiltration (Scale bar: 50 μm). FIG. 5B shows flow cytometry analysis of CD45+ T cells in treated tumors. FIG. 5C shows flow cytometry analysis of CD8+ and CD4+ T cells (gated on CD3+ T cells) in treated tumors. FIG. 5D shows the proportion of tumor-infiltrating of CD45+ T cells according to 5B (n=3). FIG. 5E shows the proportion of tumor-infiltrating of CD8+ T cells according to 5C (n=3). FIG. 5I shows the intratumoral H₂O₂ intensity test after treated with/without 8.0 wt % hydrogel in 48 h. FIG. 5G shows the proportion of intratumoral H₂O₂ intensity according to 5F (n=3). FIG. 5H shows representative H&E staining images of the treated tumors (Scale bar: 100 μm). Data depict mean±s.d., values were analyzed by one-way ANOVA with Tukey's post hoc test for 5D and 5E, and by two-tailed student's t-test for G, *P<0.05, **P<0.01, ***P<0.001.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “decrease” can refer to any change that results in a smaller gene expression, protein expression, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular hydrogel matrix comprising a chemotherapeutic agent and a blockade inhibitor is disclosed and discussed and a number of modifications that can be made to a number of molecules including the hydrogel matrix comprising a chemotherapeutic agent and a blockade inhibitor are discussed, specifically contemplated is each and every combination and permutation of hydrogel matrix comprising a chemotherapeutic agent and a blockade inhibitor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Prior chemotherapy enhances the therapeutic outcome of immunotherapy, which also reversed chemoresistance after prolonged chemotherapy. While some chemotherapeutic drugs have modest activity when used as single treatment, their combination with immunotherapy can result in enhanced anticancer effects. These observations pave the rationale to assume that some chemotherapy drugs can be used to promote an immunogenic tumor phenotype. On the other hand, engineered delivery vehicles or scaffolds are increasingly considered promising tools for transporting immunotherapeutics, with decreased systemic toxicities. However, the regulated release of payloads and the kinetics of the degradation of the supporting matrix upon in vivo administration are aspects particularly relevant for the treatment efficacy.

The past few years have witnessed exciting progresses in the immune checkpoint blockade (ICB) therapy, especially those blockading the programmed cell death protein 1/programmed cell death-ligand 1 (PD-1/PD-L1) or cytotoxic T lymphocyte antigen 4 (CTLA-4) pathway. ICB has been leveraged in treating many types of cancers, including melanoma, non-small cell lung cancer, renal cell carcinoma, urothelial carcinoma and classical Hodgkin's lymphoma.

Despite this, several challenges still need to be overcome in ICB-based cancer immunotherapy. One involves the low immune response efficacy, which is usually caused by the immunosuppressive factors, such as indoleamine-2,3-dioxygenase (IDO), interleukin-10 (IL-10), and transforming growth factor-β (TGF-β) etc. IDO, an immunosuppressive enzyme that usually overexpresses in tumors and tumor-draining lymph nodes, is one of the key issues involved in limiting T cell activation and inducing tumor immune tolerance by catalyzing the tryptophan degradation through the kynurenine pathway. As an IDO pathway inhibitor, dextro-1-methyl tryptophan (D-1MT), a tryptophan derivative that can prevent T-cell anergy triggered by IDO, has demonstrated encouraging clinical outcomes. Treatments with D-1MT have resulted in obvious regression of tumors in combination with other antitumor agents. Furthermore, the latest reports also proved that the antitumor efficacy could be remarkedly enhanced by combination of aPD-1 and D-1MT through improving the effective T cell immunity and suppressing the local IDO activity for synergistic cancer immunotherapy.

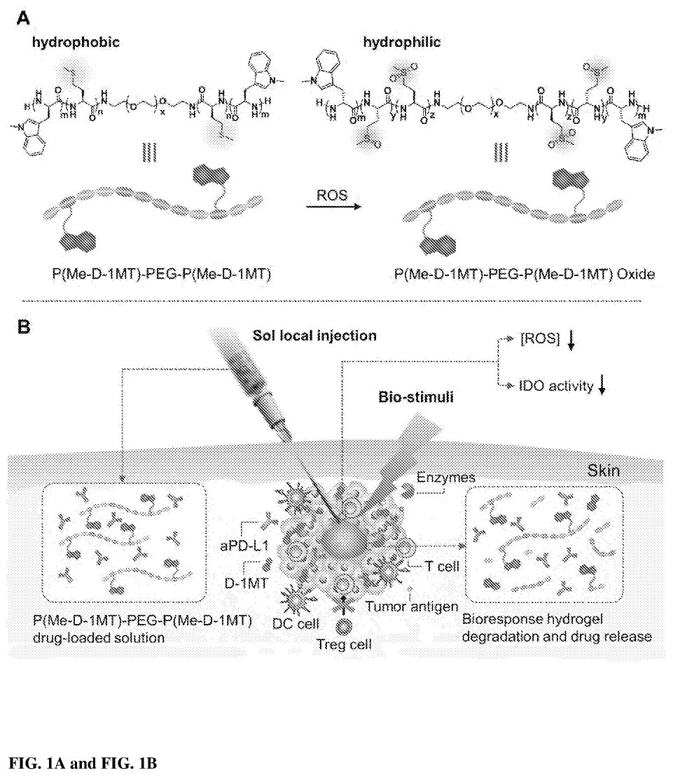

The hydrogel not only serves as a localized drug delivery depot for efficiently transporting therapeutics, but also modulates the intratumoral microenvironment for promoting effectiveness of therapy (FIG. 1). Importantly, ROS, as one of the important signaling messengers of immune system, not only is involved in many physiological processes, but also closely links with the tumor immunosuppressive microenvironment through inducing apoptosis, regulating PD-1 expression, functional suppression of T cells, as well as, promoting cancer development and progression. As a typical ROS molecule in vivo, H₂O₂ has been reported to participate in many processes, such as oxygen sensing, immune responses and cellular injuries, which also plays an essential role in carcinogenesis in vivo. It is therefore important to improve the survival of T cells and relieve the immunosuppressive tumor microenvironment by scavenging ROS including, but not limited to peroxides (for example hydrogen peroxide, organic peroxide), superoxide, hydroxyl radical, and singlet oxygen in the tumor site. ROS scavengers are known in the art and can include, for example, L-Methionine; sodium pyruvate; mannitol; sodium azide; uric acid; Ebselen; 6-hydroxy-2,5,7,8-tetramethylchroman-2-carboxylic acid (Trolox); 4,5-dihydroxybenzene-1,3-disulfonate (Tiron); α-tocopherol (Vitamin E); 2-(4-Carboxyphenyl)-4,4,5,5-tetramethylimidazoline-1-oxyl-3-oxide (Carboxy-PTIO); manganese(III)-tetrakis(4-benzoic acid)porphyrin (MnTBAP), acetyl-L-cysteine; Vitamin A; Vitamin C; Glutathione; and/or, Beta-carotene. Accordingly, in one aspect, disclosed herein are bioresponsive hydrogel matrixes comprising a reactive oxygen species scavenger (such as, for example, L-Methionine) and an inhibitor of indoleamine-2,3-dioxygenase (IDO).

As noted above, immunosuppressive factors, such as indoleamine-2,3-dioxygenase (IDO), interleukin-10 (IL-10), and transforming growth factor-β (TGF-β) etc. result in low immune response efficiency to tumors. IDO is one of the key issues involved in limiting T cell activation and inducing tumor immune tolerance by catalyzing the tryptophan degradation through the kynurenine pathway. IDO pathway inhibitors such as, for example, dextro-1-methyl tryptophan (D-1MT also known as indoximod), NLG919, BMS-986205, norharmane, rosmarinic acid, epacadostat, INCB024360 analogue, IDO inhibitor 1, PF-06840003, and/or navoximod can prevent T-cell anergy triggered by IDO. Accordingly, in one aspect, disclosed herein are bioresponsive hydrogel matrixes comprising a reactive oxygen species scavenger (such as, for example, L-Methionine) and an inhibitor of indoleamine-2,3-dioxygenase (IDO); wherein the inhibitor of IDO comprises dextro-1-methyl tryptophan (D-1MT), norharmane, rosmarinic acid, epacadostat, INCB024360 analogue, IDO inhibitor 1, PF-06840003, and/or navoximod.

As noted above, the disclosed bioresponsive hydrogel matrixes can be engineered as an injectable polypeptide-based gel depot for sustained release of immune blockade inhibitors and inhibitors of IDO, as well as ROS modulators. To facilitate these functions, the bioresponsive hydrogel was engineered as a triblock copolymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

In one aspect, the bioresponsive hydrogel can comprise a biocompatible polymer (such as, for example, methacrylated hyaluronic acid (m-HA)). In one aspect, biocompatible polymer can be crosslinked. Such polymers can also serve to slowly release the adipose browning agent and/or fat modulating agent into tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA(m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like. In one aspect, the core polymer can be flanked by polypeptide blocks. In one aspect, the flanking polypeptide blocks can comprise the ROS scavenger (such as, for example, L-Methionine, sodium pyruvate; mannitol; sodium azide; uric acid; Ebselen; Trolox; Tiron; α-tocopherol; Carboxy-PTIO; MnTBAP, acetyl-L-cysteine; Vitamin A; Vitamin C; Glutathione; and/or, Beta-carotene) and/or the inhibitor of IDO (such as, for example, D-1MT, NLG919, BMS-986205, norharmane, rosmarinic acid, epacadostat, INCB024360 analogue, IDO inhibitor 1, PF-06840003, and/or navoximod).

In one aspect, the bioresponsive hydrogel matrixes can further comprise an immune blockade inhibitor such as for example, a PD-1/PD-L1 blockade inhibitor. Herein, an injectable polypeptide-based gel depot was engineered for sustained release of aPD-L1 and D-1MT, as well as, modulating the reactive oxygen species (ROS) level in the tumor microenvironment for enhancing treatment efficacy of melanoma. The hydrogel matrix can form a micelle that encapsulates the immune blockade inhibitor. Examples, of PD-1/PD-L1 blockade inhibitors for use in the disclosed hydrogel matrixes can include any PD-1/PD-L1 blockade inhibitor known in the art, including, but not limited to nivolumab, pembrolizumab, pidilizumab, atezolizumab, avelumab, durvalumab, and BMS-936559). Thus, in one aspect, disclosed herein are hydrogel matrixes comprising a ROS scavenger, an inhibitor of IDO, and a blockade inhibitor; wherein the blockade inhibitor is a PD-1/PD-L1 blockade inhibitor such as, for example, nivolumab, pembrolizumab, spartalizumab, pidilizumab, atezolizumab, avelumab, durvalumab, and BMS-936559 or a CTLA-4 blockade inhibitor such as, for example, ipilimumab.

It is understood and herein contemplated that the disclosed bioresponse hydrogel matrixes can further comprise one or more chemotherapeutic agents. Chemotherapeutic agents that can be used in the disclosed hydrogel matrixes can comprise any chemotherapeutic known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis, (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Accordingly, in one aspect, disclosed herein are hydrogel matrixes further comprising a chemotherapeutic agent; wherein the chemotherapeutic agent is gemcitabine. In one aspect, the chemotherapeutic agent is covalently bonded to the bioresponsive hydrogel matrix.

The bioresponsive hydrogel matrixes of the present disclosure have a unique feature in that they form a stable gel at biocompatible temperatures. In one aspect, a composition comprising the bioresponsive hydrogel matrixes disclosed herein (including hydrogels comprising chemotherapeutic agents and immune blockade inhibitors) can be formulated as a solution and upon administration to the subject transition to a gel.

1. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with PD-1 and/or PD-L1 such that PD-1 is inhibited from interacting with PD-L1. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain PD-1 and/or PD-L1 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti-PD-1 and/or PD-L1 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

2. Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

3. Method of Treating Cancer and Inducing PD-1/PD-L1 Blockade Inhibitor Susceptibility in a Tumor

As noted herein, the disclosed engineered nanovesicles, engineered megakaryocytes, engineered platelets, and/or pharmaceutical compositions can be used to treat any disease where uncontrolled cellular proliferation occurs such as cancers. Accordingly, in one aspect, disclosed herein are methods of treating, reducing, inhibiting, or preventing a cancer (including, but not limited to melanoma, renal cell carcinoma, urothelial carcinoma, non-small cell lung carcinoma, and/or bladder cancer); proliferation of a cancer (including, but not limited to melanoma, renal cell carcinoma, urothelial carcinoma, non-small cell lung carcinoma, and/or bladder cancer); metastasis of a cancer (including, but not limited to melanoma, renal cell carcinoma, urothelial carcinoma, non-small cell lung carcinoma, and/or bladder cancer); and/or treating, reducing, inhibiting, or preventing relapse, proliferation or metastasis of a cancer following surgical recision of a tumor (including, but not limited to melanoma, renal cell carcinoma, urothelial carcinoma, non-small cell lung carcinoma, and/or bladder cancer) in a subject comprising administering to a patient with a cancer the engineered nanovesicle, engineered magekaryocytes, engineered platelets, and/or pharmaceutical composition disclosed herein. Thus, in one aspect, disclosed herein are methods of treating, reducing, inhibiting, or preventing a cancer; proliferation of a cancer; metastasis of a; and/or treating, reducing, inhibiting, or preventing relapse, proliferation or metastasis of a cancer following surgical recision of a tumor in a subject comprising administering to a subject a composition comprising any of the bioresponsive hydrogel matrixes disclosed herein (including for example, a bioresponsive hydrogel matrix comprising an inhibitor of IDO and a ROS scavenger). Accordingly, disclosed herein are methods of treating, reducing, inhibiting, or preventing a cancer; proliferation of a cancer; metastasis of a; and/or treating, reducing, inhibiting, or preventing relapse, proliferation or metastasis of a cancer following surgical recision of a tumor in a subject comprising administering to a subject bioresponsive hydrogel matrix comprising a reactive oxygen species scavenger and an inhibitor of indoleamine-2,3-dioxygenase (IDO).

The disclosed compositions can be used to treat any disease where uncontrolled cellular proliferation occurs such as cancers. Accordingly, in one aspect, disclosed herein are methods of treating a non-immunogenic cancer in a subject and/or inducing PD-1/PD-L1 blockade inhibitor susceptibility in a tumor in a subject with a cancer, said methods comprising administering to the subject a hydrogel matrix comprising a ROS scavenger (such as, for example L-Methionine), an inhibitor of IDO (such as, for example, D-1MT), and an immune blockade inhibitor. Examples, of PD-1/PD-L1 blockade inhibitors for use in the disclosed methods of treating a non-immunogenic cancer in a subject and/or inducing PD-1/PD-L1 blockade inhibitor susceptibility in a tumor in a subject with a cancer can include any PD-1/PD-L1 blockade inhibitor known in the art, including, but not limited to nivolumab, pembrolizumab, pidilizumab, atezolizumab, avelumab, durvalumab, and BMS-936559). Thus, in one aspect, disclosed herein are methods of treating a non-immunogenic cancer in a subject and/or inducing PD-1/PD-L1 blockade inhibitor susceptibility in a tumor in a subject with a cancer comprising administering to the subject a hydrogel matrix comprising a ROS scavenger (such as, for example L-Methionine), an inhibitor of IDO (such as, for example, D-1MT), and an immune blockade inhibitor; wherein the blockade inhibitor is a PD-1/PD-L1 blockade inhibitor such as, for example, nivolumab, pembrolizumab, pidilizumab, atezolizumab, avelumab, durvalumab, and BMS-936559.

In one aspect, the hydrogel matrix used in the disclosed methods of treating a non-immunogenic cancer in a subject and/or inducing PD-1/PD-L1 blockade inhibitor susceptibility in a tumor in a subject with a cancer comprises a chemotherapeutic agent. The chemotherapeutic used in the disclosed methods can comprise any chemotherapeutic known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

It is understood and herein contemplated that the hydrogel matrix can be designed to be bioresponsive to the microenvironment of the tumor and release the chemotherapeutic agent and/or PD-1/PD-L1 inhibitor upon exposure to factors within the microenvironment such as, for example reactive oxygen species, including, but not limited to peroxides (for example hydrogen peroxide, organic peroxide), superoxide, hydroxyl radical, and singlet oxygen the presence of acidity.

In one aspect, it is contemplated herein that the hydrogel matrix used in the disclosed methods of treating a non-immunogenic cancer in a subject and/or inducing PD-1/PD-L1 blockade inhibitor susceptibility in a tumor in a subject with a cancer can release the chemotherapeutic and PD-1/PD-L1 blockade inhibitor are released from the hydrogel at the same rate or at different rates. The hydrogel can be designed to release the chemotherapeutic and PD-1/PD-L1 blockade inhibitor into the tumor microenvironment for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. Accordingly, in one aspect, disclosed herein are methods of treating a non-immunogenic cancer in a subject and/or inducing PD-1/PD-L1 blockade inhibitor susceptibility in a tumor in a subject with a cancer can release the chemotherapeutic and PD-1/PD-L1 blockade inhibitor are released from the hydrogel for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

It is understood and herein contemplated that the disclosed methods of treating a non-immunogenic cancer in a subject and/or inducing PD-1/PD-L1 blockade inhibitor susceptibility in a tumor in a subject with a cancer can be used to treat any disease, disorder, or condition wherein uncontrolled cellular proliferation occurs such as cancers.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

A representative but non-limiting list of cancers that the disclosed compositions can be used to treat is the following: lymphoma; B cell lymphoma; T cell lymphoma; mycosis fungoides; Hodgkin's Disease; leukemias, including but not limited to myeloid leukemia; plasmacytomas; histiocytomas; bladder cancer; brain cancer, nervous system cancer, head and neck cancer, squamous cell carcinoma of head and neck, urothelial cancer, kidney cancer, lung cancers such as small cell lung cancer and non-small cell lung cancer, neuroblastoma, glioblastoma, ovarian cancer, pancreatic cancer, prostate cancer, skin cancer, liver cancer, melanoma, squamous cell carcinomas of the mouth, throat, larynx, and lung; colon cancer; cervical cancer; cervical carcinoma; breast cancer; epithelial cancer; renal cancer, genitourinary cancer; pulmonary cancer; esophageal carcinoma; head and neck carcinoma; large bowel cancer; hematopoietic cancers; testicular cancer; colon and rectal cancers; prostatic cancer; AIDS-related lymphomas or sarcomas, metastatic cancers, or cancers in general; or pancreatic cancer.

Thus, in one aspect, disclosed herein are methods of treating a cancer and/or inducing PD-1/PD-L1 blockade inhibitor susceptibility in a tumor in a subject with a cancer, wherein the cancer is a cancer with low PD-L1 expression, high PD-L1 expression, or a non-immunogenic cancer selected from the group consisting of melanoma, urothelial cancer, non-small cell lung carcinoma, renal cancer, head and neck cancer, and/or bladder cancer.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Injectable Bioresponsive Gel Depot for Enhanced Immune Checkpoint Blockade

a) Results

Herein, an injectable polypeptide-based gel depot was engineered for sustained release of aPD-L1 and D-1MT, as well as modulating the reactive oxygen species (ROS) level in the tumor microenvironment for enhancing treatment efficacy of melanoma. The hydrogel not only serves as a localized drug delivery depot for efficiently transporting therapeutics, but also modulates the intratumoral microenvironment for promoting effectiveness of therapy (FIG. 1). Importantly, ROS, as one of the important signaling messengers of immune system, not only involves in many physiological processes, but also closely links with the tumor immunosuppressive microenvironment through inducing apoptosis, regulating PD-1 express, functional suppression of T cells as well as promoting cancer development and progression. Especially, as a typical ROS molecule in vivo, H₂O₂ has been reported to participate in many processes, such as oxygen sensing, immune responses and cellular injuries, which also plays an essential role in carcinogenesis in vivo. It is therefore important to improve the survival of T cells and relieve the immunosuppressive tumor microenvironment by scavenging ROS in the tumor site.

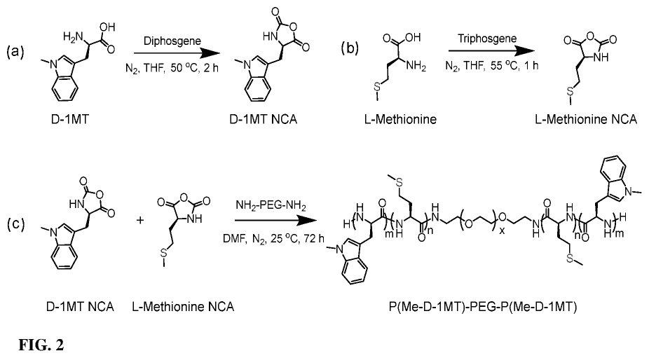

For gel construction, a functional triblock copolymer was prepared comprising a central polyethylene glycol (PEG) block flanked by two polypeptide blocks, which contains ROS-responsive L-Methionine (Me) and D-1MT (designated as P(Me-D-1MT)-PEG-P(Me-D-1MT)) (FIG. 2). The triblock copolymer was synthesized via the ring-opening polymerization (ROP) of L-Methionine N-carboxyanhydride (NCA) and D-1MT NCA by using amine-terminated PEG (Mₙ=2000) as the macromolecular initiator. The further characterization by 1H NMR indicated that the copolymer had been successfully obtained, with the polymerization degrees (DP) of L-Methionine and D-1MT at 12.0 and 1.3, respectively.

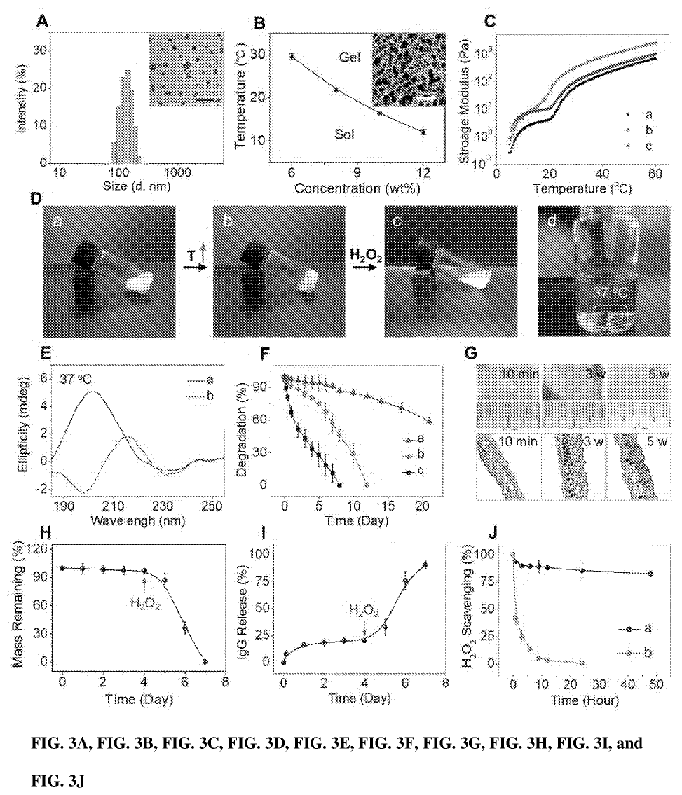

Then, the functional properties of hydrogel based on the P(Me-D-1MT)-PEG-P(Me-D-1MT) triblock copolymer were investigated by the various measurements (FIG. 3). As shown in FIG. 3A, dynamic light scattering showed that the P(Me-D-1MT)-PEG-P(Me-D-1MT) triblock copolymer can self-assemble into micelle with a hydrodynamic diameter of 130±33 nm and exhibited a spherical morphology at a relatively lower concentration (0.2 mg/mL) in aqueous solution. Nevertheless, the copolymer solution with a higher concentration (8.0 wt %) can transfer into hydrogel upon of rising temperature (FIG. 3D, a and b). And a classic thermo-responsive sol-to-gel phase transition was also observed with different concentrations in PBS (pH=7.4). As shown in FIG. 3B, the transition temperature regularly decreased from 30° C. to 12° C. with the increasing polymer concentration from 4.0 wt % to 10.0 wt %, and the porous structure of the hydrogel (8.0 wt %) after lyophilization was observed from the SEM image. The sol-to-gel transition can be caused by the thermo-driven micellar aggregation of the amphiphilic PEG-containing block copolymers. Furthermore, rheological data showed a similar trend to the phase transition results (FIG. 3C). In detail, the test sample with the concentration of 12.0 wt % (FIG. 3C, b) showed a faster gelation rate and higher storage modulus (G′, Pa), compared to the one with the lower concentration (8.0 wt %, FIG. 3C, a)). Understandably, the G′ of the 8.0 wt % hydrogel increased slightly after encapsulating a model antibody (IgG, 2.0 mg/mL), indicating that the loading of the antibody had no obvious influence to the mechanical property of the hydrogel (FIG. 3C, c).

Furthermore, rheological data showed a similar trend to the phase transition results (FIG. 3C). In detail, the test sample with the concentration of 12.0 wt % (FIG. 3C, b) showed a faster gelation rate and higher storage modulus (G′, Pa), compared to the one with the lower concentration (8.0 wt %, FIG. 3C, a)). Understandably, the G′ of the 8.0 wt % hydrogel increased slightly after encapsulating a model antibody (IgG, 2.0 mg/mL), indicating that the loading of the antibody had no obvious influence to the mechanical property of the hydrogel (FIG. 3C, c). Since the 8.0 wt % copolymer solution exhibited a suitable sol-to-gel transition temperature (around 22° C.) (FIG. 3B), ideal injectable gelation property at 37° C. (FIG. 3D, d) and good stability after gel formation (FIG. 3C), this concentration was fixed for the further biological evaluation both in vitro and in vivo.

L-Methionine (L-Me), an essential amino acid in humans, plays the key roles in mammalian metabolism in body, such as protecting some cellular organelles from oxidative stress injuries in vivo. It has been demonstrated that the poly(L-Methionine) (PMe)-based materials owned desired H₂O₂-responsive property through the oxidation of the sulfoether group into sulfoxide/sulfone. Herein was studied the H₂O₂-sensitive related properties of the copolymer which were characterized by ₁H NMR and CD, respectively. The high field moving of peaks i′ and h′ compared to the original peaks i, h indicated that the sulfoxide/sulfone groups have been generated in the oxidized copolymer. Interestingly, CD spectra (FIG. 3E) indicated that the secondary structure of the oxidized copolymer changed into a predominantly random coil that showed obvious difference compared to both the unoxidized P(Me-D-1MT)-PEG-P(Me-D-1MT) and the reported oxidized PMe-based polymers. Moreover, the FTIR spectra also validated the transition from a predominantly β-sheet conformation to a mainly random coil structure with the generation of sulfoxide/sulfone groups, after the oxidation of the triblock copolymer with H₂O₂. These can be caused by the D-1MT component which endowed with an additional hydrophobicity and dextral chiral property.

The degradation behavior of the formed gel was investigated both in vitro and in vivo. According to FIG. 3F, the gel erosion mainly drove the weight loss of the formed gel in Tris-HCl buffer solution (pH 7.4)). A mass loss around 40% was observed in three weeks. Significantly, the degradation rate of the hydrogel can be enhanced by the stimuli of proteinase K (5.0 U/mL) or H₂O₂ (2.0 mM) in Tris-HCl (pH=7.4). The hydrogel degraded completely in 8 days and 12 days in the presence of proteinase K and H₂O₂, respectively. The in vivo degradation test indicated that this copolymer aqueous solution (50 μL, 8.0 wt %) can quickly transfer into hydrogel within a few minutes after subcutaneous injection toward mice. The following study demonstrated that the hydrogel possessed good biodegradability in vivo, which showed obvious reduction in volume after 3 weeks and dissolved in around 5 weeks weeks after injection (FIG. 3G).

Furthermore, the above solution associated with the degraded gel was also collected and analyzed by HPLC to evaluate the release behavior of D-1MT in vitro. Without proteinase K, only around 1.0% of D-1MT was released from the gel within 8 days, but an enhanced cumulative release of D-1MT (9.6%) was observed with the presence of proteinase K during the same period. Despite D-1MT was released from the gel at a relatively slow rate, an obvious pattern of inhibiting kynurenine production was still observed for the degradation fragments of the hydrogel. This indicated that the D-1MT containing degradation fragments of P(Me-D-1MT)-PEG-P(Me-D-1MT) retained the IDO inhibiting activity, and the relatively lower inhibition efficacy of the triblock copolymer compared to free D-1MT can be caused by the sustained release of D-1MT.

To evaluate the ROS-responsive cargo release profile, IgG was loaded into the hydrogel as a model antibody, and the release behavior was detected with or without the presence of H₂O₂ in vitro (FIG. 3H). It was found that the drug-loaded hydrogel kept a relatively stable status with a slow mass loss in PBS (pH=7.4) during the first 4 days, but an obviously increased degradation rate was triggered by H₂O₂ (10 mM), with an entire degradation of the gel in 3 days. Accordingly, only around 20% of IgG was released from the hydrogel in the first 4 days without H₂O₂. Whereas over 90% IgG was released from the system after addition of H₂O₂ (10 mM) during the following 3 days, indicating that H₂O₂ can trigger and accelerate the drug release from the hydrogel (FIG. 3I). Besides, this gel also showed excellent H₂O₂ scavenging ability which eliminated around 60% of H₂O₂ in 1 hour and almost completely cleaned up H₂O₂ in 24 hours (FIG. 3J).

Moreover, 3-(4,5-dimethyl-thiazol-2-yl)-2,5-diphenyl tetrazolium bromide (MTT) results showed that above 95% of the cells remained viable after incubated with the gel (8.0 wt %) at various concentrations, indicating that this material possessed good cytocompatibility. Also, the hematoxylin-eosin staining (H&E) images of the host skin tissues around the injection site surrounding the hydrogel showed no evident chronic inflammatory reaction during or after degradation of the hydrogel (FIG. 3G).

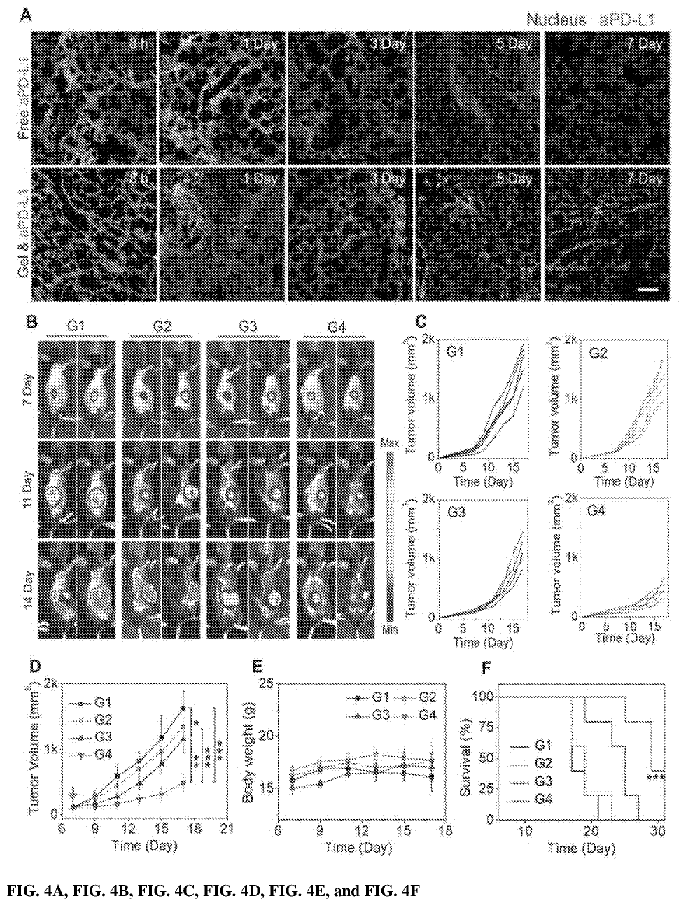

To assess the potential of this hydrogel as a drug delivery depot, the intratumoral drug release behavior and antitumor efficacy were evaluated on the B16F10 tumor bearing C57BL6 female mice. shown in FIG. 4A, both the free aPD-L1 (2.0 mg/kg) and aPD-L1-loaded gel showed a high intratumoral fluorescence intensity at the first 8 h after intratumoral injection. However, remarkable attenuation of the green fluorescence intensity for the group treated with free aPD-L1 was observed on day 3 after injection. Almost no visible green fluorescence signals was observed on day 7 after treatment. By contrast, a stronger green fluorescence intensity was found in the group treated with aPD-L1-loaded hydrogel, and green fluorescence signals were still detected on day 7 after treatment. These data proved that the polypeptide-based hydrogel can markedly prolong the retention time of the protein cargo in the tumor site.

To further evaluate the synergistic immune antitumor efficiency, melanoma-bearing female C57BL6 mice were randomly grouped and treated with a single intratumoral injection of PBS (G1), blank P(Me-D-1MT)-PEG-P(Me-D-1MT) hydrogel (G2), Free aPD-L1 (2.0 mg/Kg) and D-1MT (4.5 mg/Kg) (G3) and aPD-L1-loaded P(Me-D-1MT)-PEG-P(Me-D-1MT) hydrogel (aPD-L1 2.0 mg/Kg, D-1MT 4.5 mg/Kg) (G4) when the tumor volumes reached to ˜110 mm³ on the 7^{th} day, respectively. In the following 10 days, the tumor growth was monitored with the bioluminescence signal of B16F10-luc cells (FIG. 4B). It was shown that the tumor growth of the mice treated with aPD-L1-loaded hydrogel (G4) was distinctly delayed than the other three groups (G1-G3). However, the blank hydrogel treated group (G2) only showed a little tumor inhibition effect compared to the PBS treated group (G1), which can be caused by the delayed release behavior of D-1MT due to the slow degradation rate of the hydrogel. Notably, the tumor growth of free drug-treated group was markedly inhibited in the first treated 4 days, but the tumor growth rate gradually accelerated in the following 6 days (FIGS. 4, C and D). This result can be caused by the rapid diffusion and metabolism of the free drug in body which could not meet the efficacy drug dose after treated 3 days (FIG. 4A). Furthermore, the body weight showed no significant loss after various treatments (FIG. 4E). And the mouse survival rate of G4 was markedly improved compared to the other three groups within the observed 31 days (FIG. 4F).

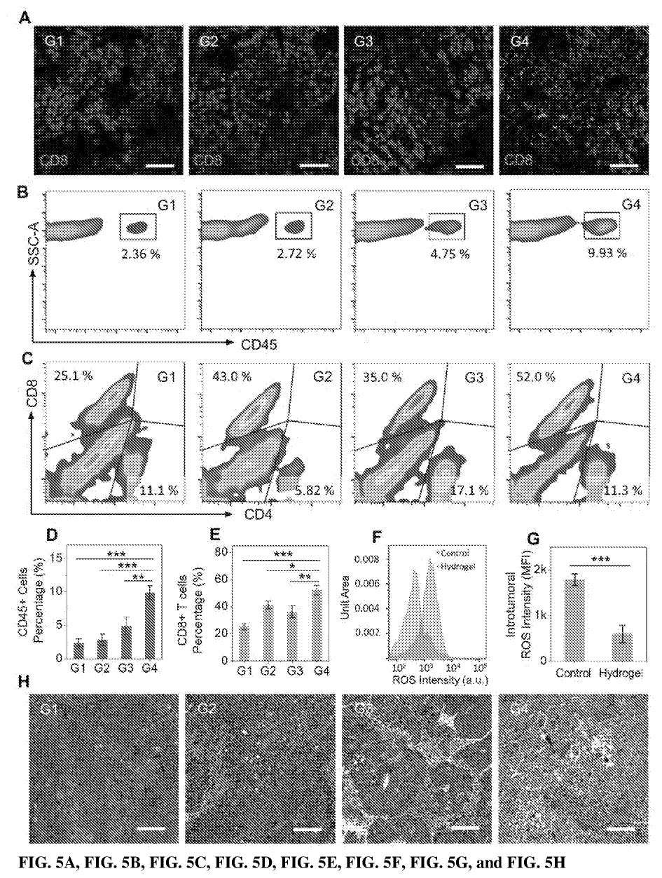

To investigate the infiltration behavior of immune cells in the tumor site after treatments, the tumor-infiltrating lymphocytes (TILs) were collected from the tumor tissues and analyzed by the immunofluorescence and flow cytometry 10-days post-treatment._{[25]} The immunofluorescence images indicated that there were limited T cell infiltration in the tumor of PBS treated control group (FIG. 5A). Contrarily, the other three treated groups showed higher intensity of CD8+ T cells compared to the control group. The tumors treated with aPD-L1-loaded hydrogel (G4) was obviously infiltrated with CD8+ T cells. Furthermore, the tumors which treated with aPD-L1-loaded hydrogel (G4) also displayed the highest immune cell ratio compared to the other three groups (FIGS. 5, B and D). The percentage of CD8+ T cells in the tumors treated with aPD-L1-loaded hydrogel (G4) was over 2-fold of that in the PBS-treated group (G1) and around 1.6-fold compared to the group treated with free drugs (G3) (FIGS. 5, C and E). Notably, the free gel treated group showed a higher CD8+ T cell infiltration compared to the treated with free drugs. This difference can be caused by the tumor microenvironmental regulation of the free gel through IDO-inhibition and ROS scavenge (FIGS. 5, F and G). Collectively, these data indicated that the combination therapy by localized delivering aPD-L1 and D-1MT through P(Me-D-1MT)-PEG-P(Me-D-1MT) hydrogel can cause an effective T-cell-mediated immune response.

The hematoxylin/eosin (H&E) staining images also validated that the group treated with aPD-L1-loaded hydrogel presented the highest extent of tumor cell death compared to the other groups. Meanwhile, the H&E images of the main organs, including liver, lung, kidney, spleen and heart, showed negligible damage or inflammation except the spleen image of G3, which showed some pathological change. The pathological change can be caused by the peak dosage toxicity of the free drugs. In conclusion, a new thermogelling ROS-responsive hydrogel-based localized drug delivery platform was generated for combination cancer immunotherapy that pertinently inhibited the immunoinhibitory ligand PD-L1 and suppressed immunosuppressive enzyme IDO activity for the enhancement of antitumor immune response. The biocompatible P(Me-D-1MT)-PEG-P(Me-D-1MT)-based hydrogel could not only sustain deliver aPD-L1 and D-1MT in situ, but also decline the intratumoral ROS level. The in vivo study demonstrated that the aPD-L1-loaded hydrogel naturally stimulated infiltration of immune cells and enhance the antitumor efficacy compared to the free drugs at a comparable dose. This thermogelling polypeptide hydrogel holds promise as a localized drug delivery platform for enhancing cancer immunotherapy in a simple administration manner.

b) Materials and Methods

(1) Materials.

Amine-PEG-Amine (Mr=2000) was purchased from Laysan bio. Inc. Dextro-1-methyltryptophan (D-1MT) was obtained from Cayman Chemical Inc. Proteinase K (Recombinant, PCR grade), Cellular Reactive Oxygen Species Detection Assay Kit (Deep Red Fluorescence) and most solvent were purchased from Thermo Fisher Scientific Inc. 3-(4,5-dimethyl-thiazol-2-yl)-2,5-diphenyl tetrazolium bromide (MTT), GoInVivo™ Purified anti-mouse CD274 antibody (B7-H1, aPD-L1), Cy3 Goat anti-rat IgG (minimal x-reactivity) Antibody, human recombinant IFN-γ, Alexa Fluor 488 anti-mouse CD8a and APC anti-mouse CD4 were obtained from Biolegend Inc. Rat IgG total ELISA kit was purchased from affymetrix, Inc. Fluorimetric Hydrogen Peroxide Assay Kit, L-Glutamic acid, Sodium azide L-Methionine and other chemicals were obtained from Sigma-Aldrich Co. The human cervical cancer cell line (HeLa) was purchased from the American Type Culture Collection (ATCC). The B16F10-Luc cell line was obtained from Dr. Leaf Huang's lab at UNC-CH. Female C57BL6 mice (5˜6 week-old) were obtained from Jackson Lab (USA). All the mouse studies were performed in the context of the animal protocol approved by the Institutional Animal Care and Use Committee at the University of North Carolina at Chapel Hill and North Carolina State University.

(2) Synthesis and Characterization of the P(Me-D-1MT)-PEG-P(Me-D-1MT) Polypeptides.

D-1MT NCA and L-Methionine NCA were obtained according to the previous work (J. Am. Chem. Soc. 2014, 136, 5547). Then these NCA were used to synthesize P(Me-D-1MT)-PEG-P(Me-D-1MT) copolymer using our reported methods with some changes (Adv. Healthcare Mater. 2016, 5, 1979). Briefly, NH₂-PEG-NH₂ 1.0 g (0.5 mmol) was dried through azeotropic distillation for 3 hours in toluene at 110° C. Then the solvent was evaporated and the anhydrous DMF (25 mL) was added to the bottle to re-dissolve NH₂-PEG-NH₂. Both the D-1MT NCA (0.245 g, 1.0 mmol) and L-Methionine NCA (1.75 g, 10 mmol) was added to the reaction system with N₂ protection after NH₂-PEG-NH₂ was dissolved completely. The reaction was stirred for 72 h under N₂ atmosphere at the room temperature. Then, the reaction mixture was dialyzed against water for 72 h at room temperature. The final polymer was obtained after lyophilization, and the structures of compounds were determined by ¹H NMR (Varian Gemini 2300).

(3) The Secondary Structure Study.

The secondary structure of both the P(Me-D-1MT)-PEG-P(Me-D-1MT) and P(Me-D-1MT)-PEG-P(Me-D-1MT) oxide with the concentration of 0.1 mg/mL were analyzed by CD spectrometer (Aviv) at different temperature in PBS (pH=7.4).

(4) Dynamic Light Scattering (DLS).

The self-assemble behaviors of the material with or without H₂O₂ were investigated on a Zetasizer (Nano ZS, Malven) in water. 0.25 mg/mL of P(Me-D-1MT)-PEG-P(Me-D-1MT) and P(Me-D-1MT)-PEG-P(Me-D-1MT) oxide were dissolved in water and stirred for three days, respectively. Then, the samples were pre-treated with the filter (0.45 μm) and determined by the Zetasizer.

(5) Transmission Electron Microscopy (TEM).

The P(Me-D-1MT)-PEG-P(Me-D-1MT) aqueous solution with the concentration of 0.1 mg/mL was dropped on the TEM copper grid (300 mesh) and dried in air. Then, TEM (JEM-2000FX, Hitachi) was used to collect the information of the sample.

(6) The Sol-Gel Phase Test.

The P(Me-D-1MT)-PEG-P(Me-D-1MT) was dissolved in 1.0 mL PBS at different concentrations (4.0 wt %, 6.0 wt %, 8.0 wt % and 10.0 wt %) and stirred in ice/water bath for 48 h. After that, 300 μL of the material solution was moved into little vials which owned 8 mm inner diameter. Then, the sol-gel transfer behavior was characterized by the test tube inverting method through increasing the temperature of 1° C. per step in every 10 min. And the temperature will be recorded as the sol-gel transition point when there no fluidity was observed in 30 s after the test vial. Triplicate tests were carried for each data point.

(7) Micellization Behaviors.

To investigate the micellization behavior of the material, an aqueous solution of P(Me-D-1MT)-PEG-P(Me-D-1MT) with a concentration of 0.1 mg/mL was prepared and determined on a Malvern Zetasizer NanoZS at 37° C., and the TEM image of aggregate's morphology was also characterized on a JEOL 2000FX TEM instrument at 200 kV.

(8) Scanning Electron Microscopy (SEM).

The microstructure of the hydrogel was observed on a FEI Verios 460L field emission scanning electron microscopy (FESEM, 20 kV), which the sample (8.0 wt %) was dried by freeze dryer after repaid freezing in liquid nitrogen.

(9) The Rheology Properties Test.

The variation of thermo-dependent rheology properties of the P(Me-D-1MT)-PEG-P(Me-D-1MT) in different concentrations with/without IgG (1.0 mg/mL) were recorded on an MCR 301 rheometer of Anton Paar during the sol-gel transitions with the hating rate in 0.5° C. min⁻¹.

(10) Hydrogel Degradation Behaviors and D-1MT Release.

P(Me-D-1MT)-PEG-P(Me-D-1MT) hydrogel with the concentration of 8.0 wt % (300 μL) was formed in a vail with an inner dimeter of 1.0 cm at 37° C. for 15 min. The tris-HCl buffer solution with 0 mM, 2.0 mM, 10 mM H₂O₂ or with proteinase K (5 U/mL) were used as the degradation media (pH=7.4), respectively. All the samples were incubated at 37° C. with gentle shaking. The media was moved out and kept in 4° C. and the fresh media was added after the gel remaining mass of every sample was recorded in the designed time intervals. Furtherly, the structure of released D-1MT in the gel leaching solution was confirmed by HPLC-MS, and the release rate was analysed by HPLC with ammonium acetate buffer/acetonitrile (92:8) as the mobile phase. The D-1MT contents were determined at an excitation wavelength of 280 nm.

(11) H₂O₂ Reduce Rate Test.

The hydrogel was formed in a vail with 300 μL P(Me-D-1MT)-PEG-P(Me-D-1MT) solution (8.0 wt %), then 2 mL H₂O₂ solution (10 mM) was added into the vail and incubated in 37° C., and the free H₂O₂ solution (10 mM) as control. At different interval, 10 μL sample was transferred out and kept at −20° C., and fresh PBS with the corresponding volume was added to the system. All the samples were detected by a Fluorimetric Hydrogen Peroxide Assay Kit, and triplicate tests were carried for each data point.

(12) IgG Release Test In Vitro.

0.3 mg Immunoglobulin G antibody (IgG, 1.0 mg/mL) was used as a model drug and added into 300 μL copolymer solution (8.0 wt %) for each vial. The samples were incubated on an orbital shaker at 37° C. and 3.0 mL PBS (pH=7.4) with different H₂O₂ concenration was used as the drug release media. At the desired interval, all the media of the sample was removed and stored at −20° C. for further analysis and another 3.0 mL fresh media was then added to the vial. The released amount of IgG was measured by a rat IgG ELISA. The absorbance was detected by a UV-vis spectrophotometer at 450 nm and diluted free antibody as a standard curve.

(13) IDO Cellular Activity Test.

The IDO enzyme inhibition assay was investigated according to the previous method with little modifiation (ACS Nano 2016, 10, 8956). Briefly, HeLa cells were seeded with the density of 5×10⁴ cells/well (12-well plate) in 2.0 mL DMEM which contained 100 μM L-tryptophan. Then, free D-1MT and P(Me-D-1MT)-PEG-P(Me-D-1MT) were added into the wells at the designated concentration next day. Thereafter, IFN-γ with the final concentration of 0.1 μg/mL was added into each well to stimulate the IDO expression. After 72 h incubation, 200 μL supernatants were moved out and deposited in a new 96-well plate and 10 μL 30% TCA was added into every well for protein precipitation. The produced kynurenine was analyzed by HPLC with ammonium acetate buffer/acetonitrile (92:8) as the mobile phase and the excitation wavelength fixed at 360 nm. The experiment was repeated three times.

(14) Cytotoxicity Evaluation In Vitro.

Both the B16F10 and HeLa cell lines were used to evaluate the relative cytotoxicity of the hydrogel at different concentrations. Briefly, 1×10⁴ cells were seeded in each well in 24-well plates with overnight incubating in DMEM (1 mL). Different volumes of the P(Me-D-1MT)-PEG-P(Me-D-1MT) solution (8.0 wt %) were dropped on the transwell and incubated 10 mM at 37° C. After that, the hydrogel loaded transwell was moved to the cell-seeded plates and co-incubated for another 48 h. Then, the transwell was moved out, and the cell viability was evaluated by MTT assay. Each data point was measured for three times.

(15) The Degradation and Biocompatibility Study In Vivo.

50 μL P(Me-D-1MT)-PEG-P(Me-D-1MT) solution with the concentration of 8.0 wt % in PBS was injected into the right flank of C57 mice. At the determined time points (30 min, 3 weeks and 5 weeks), the mice were sacrificed, and the gel status was recorded, and the skins attached to the hydrogels were collected and kept in 4.0% (w/v) paraformaldehyde with 1×PBS at 4° C. Then H&E staining slices of the tissues were prepared and observed with a microscope.

(16) Tumor Models In Vivo.

In this work, all the mouse studies were performed in the context of the animal protocol approved by the Institutional Animal Care and Use Committee at the University of North Carolina at Chapel Hill and North Carolina State University. Briefly, 1.5×10⁶ B16F10 cells suspension in 50 μL PBS was transplanted into the right flank of the C57B6 female mice. When the tumor volumes reached to ˜110 mm³, the following experiments were carried out in details.

(17) Intratumoral ROS Intensity Test.

20 μL PBS or P(Me-D-1MT)-PEG-P(Me-D-1MT) solution (8.0 wt %, PBS) were injected into the tumors mildly. After 48 h, the mice were sacrificed and the tumor tissues were collected and kept on ice for the further research. Then, the intratumoral ROS intensity was determined by a Cellular Reactive Oxygen Species Detection Assay Kit (Deep Red Fluorescence) and followed the commercial protocol.

(18) Intratumoral Drug Release Test.

20 μL PBS or P(Me-D-1MT)-PEG-P(Me-D-1MT) solution (8.0 wt %, PBS) which contained aPD-L1 (2.0 mg/mL) were injected into the melanoma tumor. And then the tumors were collected and stored at different intervals respectively. Thereafter, tumor frozen sections were stained by DAPI and Cy3 labelled secondary antibody overnight at 4° C. after blocking with BSA (3.0%). then the fluorescence images were collected by CLSM (Ziss 710).

(19) Antitumor Evaluation In Vivo.

Mice were divided into four groups randomly after weighted. Different drug formulations (20 μL) were injected into tumors carefully and different experiments were carried out as follows: PBS (G1), Injectable P(Me-D-1MT)-PEG-P(Me-D-1MT) hydrogel (G2), Free D-1MT and aPD-L1 (G3), and aPD-L1 loaded P(Me-D-1MT)-PEG-P(Me-D-1MT) hydrogel (G4). The tumor size and mice weight were monitored every two days after the treatments. The tumor volume was calculated with the formula V=½a×b², which a and b represent the length and width of the tumor, respectively. After treatment, histopathology was analyzed on the tumor and major organs (liver, heart, lung, spleen, kidney, etc.).

### D. REFERENCES

A. Gras Navarro, A. T. Bjorklund, M. Chekenya, Front. Immunol. 2015, 6, 202; G. T. Motz, G. Coukos, Immunity 2013, 39, 61; D. L. Xie, J. Wu, Y. L. Lou, X. P. Zhong, PNAS 2012, 109, 14152.
A. R. Rodriguez, J. R. Kramer, T. J. Deming, Biomacromolecules 2013, 14, 3610.
C. A. Opitz, U. M. Litzenburger, U. Opitz, E Sahm, K. Ochs, C. Lutz, W. Wick, M. Platten, PLOS one 2011, 6, e19823; A. C. Moreno, R. O. Clara, J. B. Coimbra, A. R. Julio, R. C. Albuquerque, E. M. Oliveira, S. S. Maria-Engler, A. Campa, FEBS J. 2013, 280, 4782.
C. Wang, W. Sun, Y. Ye, Q. Hu, H. N. Bomba, Z. Gu, Nat. Biomed. Eng. 2017, 1, 0011.
C. Wang, Y. Ye, G. M. Hochu, H. Sadeghifar, Z. Gu, Nano Lett. 2016, 16, 2334.
D. Trachootham, J. Alexandre, P. Huang, Nat. Rev. Drug Discov. 2009, 8, 579; C. Wang, J. Wang, X. Zhang, S. Yu, D. Wen, Q. Hu, Y. Ye, H. Bomba, X. Hu, Z. Liu, G. Dotti, Z. Gu, Sci. Transl. Med. 2018, 10, eaan3682.
D. Y. Hou, A. J. Muller, M. D. Sharma, J. DuHadaway, T. Banerjee, M. Johnson, A. L. Mellor, G. C. Prendergast, D. H. Munn, Cancer Res. 2007, 67, 792; S. G. Awuah, Y R. Zheng, P. M. Bruno, M. T. Hemann, S. J. Lippard, J. Am. Chem. Soc. 2015, 137, 14854.
J. Moskovitz, S. Bar-Noy, W. M. Williams, J. Requena, B. S. Berlett, E. R. Stadtman, PNAS 2001, 98, 12920; M. L. Martinez-Chantar, F. J. Corrales, L. A. Martinez-Cruz, E. R. Garcia-Trevijano, Z. Z. Huang, L. Chen, G. Kanel, M. A. Avila, J. M. Mato, S. C. Lu, FASEB J 2002, 16, 1292.
J. R. Kramer, T. J. Deming, J. Am. Chem. Soc. 2012, 134, 4112.
K. M. Mahoney, P. D. Rennert, G. J. Freeman, Nat. Rev. Drug Discov. 2015, 14, 561.
L. Chen, X. Han, J. Clin. Invest. 2015, 125, 3384; W. Zou, J. D. Wolchok, L. Chen, Sci. TransL Med. 2016, 8, 328rv4.
L. Yin, H. Tang, K. H. Kim, N. Zheng, Z. Song, N. P. Gabrielson, H. Lu, J. Cheng, Angew Chem. Int. Ed. 2013, 52, 9182.
L. Yu, H. Zhang, J. Ding, Angew Chem. Int. Ed. 2006, 45, 2232.
M. K. Joo, D. Y. Ko, S. J. Jeong, M. H. Park, U. P. Shinde, B. Jeong, Soft Matter 2013, 9, 8014.
N. Qiu, X. Liu, Y. Zhong, Z. Zhou, Y. Piao, L. Miao, Q. Zhang, J. Tang, L. Huang, Y. Shen, Adv. Mater 2016, 28, 10613; X. Z. Yang, X. J. Du, Y. Liu, Y. H. Zhu, Y. Z. Liu, Y. P. Li, J. Wang, Adv. Mater. 2014, 26, 931.
Q. Xu, C. He, K. Ren, C. Xiao, X. Chen, Adv. Healthcare Mater 2016, 5, 1979.
S. G. Rhee, Science 2006, 312, 1882; B. C. Dickinson, C. J. Chang, Nat. Chem. Biol. 2011, 7, 504; M. Reth, Nat. Immunol. 2002, 3, 1129; S. Reuter, S. C. Gupta, M. M. Chaturvedi, B. B. Aggarwal, Free Radic. Biol. Med. 2010, 49, 1603; Q. Chen, C. Liang, X. Sun, J. Chen, Z. Yang, H. Zhao, L. Feng, Z. Liu, PNAS 2017, 114, 5343.
S. Lob, A. Konigsrainer, H. G Rammensee, G. Opelz, P. Terness, Nat. Rev. Cancer 2009, 9, 445; T. Inaba, K. Ino, H. Kajiyama, E. Yamamoto, K. Shibata, A. Nawa, T. Nagasaka, H. Akimoto, O. Takikawa, F. Kikkawa, Gynecol. Oncol. 2009, 115, 185; C. A. Opitz, U. M. Litzenburger, F. Sahm, M. Ott, I. Tritschler, S. Trump, T. Schumacher, L. Jestaedt, D. Schrenk, M. Weller, M. Jugold, G. J. Guillemin, C. L. Miller, C. Lutz, B. Radlwimmer, I. Lehmann, A von Deimling, W. Wick, M. Platten, Nature 2011, 478, 197.
S. Yu, D. Zhang, C. He, W. Sun, R. Cao, S. Cui, M. Deng, Z. Gu, X. Chen, Biomacromolecules 2017, 18, 4341.
W. Dröge, Physiol. Rev. 2002, 82, 47; K. C. Sheng, M. D. Wright, V. Apostolopoulos, Curr. Med. Chem. 2011, 18, 5507; L.-Y. OuYang, X.-J. Wu, S.-B. Ye, R.-x. Zhang, Z.-L. Li, W. Liao, Z.-Z. Pan, L.-M. Zheng, X.-S. Zhang, Z. Wang, Q. Li, G. Ma, J. Li, J. Transl. Med. 2015, 13, 47; Laura A. Sena, Navdeep S. Chandel, Molecular Cell 2012, 48, 158; Y. Liu, J. Wei, G. Guo, J. Zhou, Immunopharm. Immunot. 2015, 37, 359; V. Tkachev, S. Goodell, A. W. Opipari, L. Y. Hao, L. Franchi, G. D. Glick, J. L. Ferrara, C. A. Byersdorfer, J. Immunol. 2015, 194, 5789.
W. Zou, Nat. Rev. Cancer 2005, 5, 263; C. Ghirelli, T. Hagemann, J. Clin. Invest. 2013, 123, 2355.
X. Chen, M. Song, B. Zhang, Y. Zhang, Oxid. Med. Cell Longev. 2016, 2016, 1580967.
Y. Cheng, C. He, C. Xiao, J. Ding, X. Zhuang, Y. Huang, X. Chen, Biomacromolecules 2012, 13, 2053.
Y. Cheng, C. He, J. Ding, C. Xiao, X. Zhuang, X. Chen, Biomaterials 2013, 34, 10338.
Y. Shen, X. Fu, W. Fu, Z. Li, Chem. Soc. Rev. 2015, 44, 612.
Y. Ye, J. Wang, Q. Hu, G. M. Hochu, H. Xin, C. Wang, Z. Gu, ACS Nano 2016, 10, 8956.

1. A bioresponsive hydrogel matrix comprising a reactive oxygen species scavenger and an inhibitor of indoleamine-2,3-dioxygenase (IDO).

2. The bioresponsive hydrogel matrix of claim 1, wherein the hydrogel matrix is a triblock copolymer.

3. The bioresponsive hydrogel matrix of claim 2, wherein the triblock copolymer comprises polyethylene glycol flanked by a polypeptide block comprising the reactive oxygen species scavenger and an inhibitor of indoleamine-2,3-dioxygenase (IDO).

4. The bioresponsive hydrogel matrix of claim 1, wherein the reactive oxygen species scavenger comprises L-Methionine.

5. The bioresponsive hydrogel matrix of claim 1, wherein the inhibitor of IDO comprises dextro-1-methyl tryptophan (D-1MT), norharmane, rosmarinic acid, epacadostat, INCB024360 analogue, IDO inhibitor 1, PF-06840003, or navoximod.

6. The bioresponsive hydrogel matrix of claim 1 further comprising an immune blockade inhibitor.

7. The bioresponsive hydrogel matrix of claim 6, wherein the immune blockade inhibitor comprises a PD-1/PD-L1 blockade inhibitor

8. The bioresponsive hydrogel matrix of claim 7, wherein the PD-1/PD-L1 blockade inhibitor is selected from the group consisting of nivolumab, pembrolizumab, pidilizumab, atezolizumab, avelumab, durvalumab, and BMS-936559.

9. The bioresponsive hydrogel matrix of claim 1 further comprising a chemotherapeutic agent.

10. A method of treating a cancer in a subject comprising administering to the subject the bioresponsive hydrogel matrix of claim 1.

11. A method of treating a cancer in a subject comprising administering to the subject a bioresponsive hydrogel matrix comprising a reactive oxygen species scavenger and an inhibitor of indoleamine-2,3-dioxygenase (IDO).

12. The method of claim 11, wherein the reactive oxygen species scavenger comprises L-Methionine.

13. The method of claim 11, wherein the inhibitor of IDO comprises dextro-1-methyl tryptophan (D-1MT), norharmane, rosmarinic acid, epacadostat, INCB024360 analogue, IDO inhibitor 1, PF-06840003, or navoximod.

14. The method of claim 1, wherein the bioresponsive hyrodrogel matrix further comprises an immune blockade inhibitor.

15. The method of claim 14, wherein the immune blockade inhibitor comprises a PD-1/PD-L1 blockade inhibitor.

16. The method of claim 15, wherein the PD-1/PD-L1 blockade inhibitor is selected from the group consisting of nivolumab, pembrolizumab, pidilizumab, atezolizumab, avelumab, durvalumab, and BMS-936559.

17. The method of claim 1, wherein the bioresponsive hyrodrogel matrix further comprises a chemotherapeutic agent.

18. The method of claim 1, wherein the hydrogel matrix releases the inhibitor of IDO, the immune blockade inhibitor, the chemotherapeutic agent, or any combination thereof into the tumor microenvironment upon exposure to reactive oxygen species (ROS).

19. The method of claim 18, wherein the hydrogel releases the chemotherapeutic and PD-1/PD-L1 blockade inhibitor into the tumor microenvironment for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

20. The method of claim 1, wherein the cancer is a cancer selected from the group consisting of melanoma, non-small cell lung carcinoma, urothelial cancer, renal cancer, head and neck cancer, Hodgkin's lymphoma, and bladder cancer.
